(19) United States
(12) Patent Application Publication
(10) Pub. No.: US 2023/0009148 A1
(43) Pub. Date: Jan. 12, 2023

(54) An Anti-Microbial Coated Urethral Plug for CAUTI Reduction

(71) Applicant: C. R. Bard, Inc., Franklin Lakes, NJ (US)

(72) Inventor: Pranav Challa, Wood-Ridge, NJ (US)

(21) Appl. No.: 17/783,857

(22) PCT Filed: Nov. 24, 2020
(86) PCT No.: PCT/US20/62063

§ 371 Date: Jun. 9, 2022

Related U.S. Application Data
(60) Provisional application No. 62/946,871, filed on Dec. 11, 2019.

(51) Int. Cl. A61M 25/00

(52) U.S. Cl. CPC A61M 25/0017; A61M 2025/0056; A61M 2025/006

(57) ABSTRACT

Embodiments disclosed herein are directed to an anti-microbial coated urethral plug to reduce catheter associated urinary tract infections (“CAUTI”). A urethral plug is configured to engage a urinary catheter and includes a plug body defining a frustum shape, a lumen configured to receive the urinary catheter, and a slit extending longitudinally and extending from the lumen to a side surface of the plug body. The plug can further include one of a textured surface and a coating disposed on an outer surface and configured to mitigate microbial adhesion. A distal portion of the plug is configured to fit within a urethral opening to create a seal between the side surface of the plug body and the urethral opening. The urethral plug includes coatings, textured surfaces, or the like, and provides protection against the extra-luminal migration of microbes that may cause CAUTI.

### PRIORITY

This application claims the benefit of priority to U.S. Provisional Application No. 62/946,871, filed Dec. 11, 2019, which is incorporated by reference in its entirety into this application.

### SUMMARY

Briefly summarized, embodiments disclosed herein are directed to an anti-microbial coated urethral plug to reduce catheter associated urinary tract infections (“CAUTI”). Hospital Acquired Infections (“HAI”), such as CAUTI, are detrimental to the patient, and also incur extra costs in treating these additional complications. Two thirds of all CAUTI occur due to extra-luminal bacterial migration, or bacteria that climb the outside of the Foley catheter into the urinary tract. Some urinary catheters currently available can include various coatings, which when wetted, have anti-microbial properties and can mitigate CAUTI. These coatings are disposed over substantially the entire length of the catheter but can also drive up costs of such devices. These additional costs can deter usage, allowing HAI, such as CAUTI, to persist.

Disclosed herein are apparatus and methods for applying a plug to the urinary meatal opening, after insertion of a urinary catheter, to create a seal therebetween and mitigate extra-luminal bacterial migration. The plug includes coatings, textured surfaces, combinations thereof, or the like, to further mitigate microbial migration at the urethral entrance. Advantageously, embodiments disclosed herein can be used in conjunction with coated urinary catheters to provide improved protection against CAUTI. Further, embodiments disclosed herein can be used with uncoated catheters to reduce costs, improving usage uptake, and still provide effective protection against CAUTI.

Embodiments disclosed herein include a urethral plug configured to engage a urinary catheter and to mitigate urinary tract infection. The urethral plug includes a plug body defining a frustum shape and has a lumen configured to receive the urinary catheter, a slit extending longitudinally and extending from the lumen to a side surface of the plug body, the slit configured to allow the urinary catheter to pass therethrough, one of a textured surface and a coating disposed on a portion of an outer surface and configured to mitigate microbial adhesion, and a distal portion configured to fit within a urethral opening to create a seal between the side surface of the plug body and the urethral opening.

In some embodiments, the urethral plug further includes a proximal portion configured to remain outside of the urethral opening. The urethral plug further includes a proximal end defining a first diameter, and a distal end defining a second diameter, the second diameter being less than the first diameter. The first diameter is between 3 mm and 30 mm, and the second diameter is between 2 mm and 10 mm. The first diameter is 10 mm, and the second diameter is 4 mm. The textured surface includes one of a superomniphobic texture, a nano-cone texture, a superhydrophobic coating, a Manganese oxide polystyrene (MnO₂/PS) nano-composite, a Zinc oxide polystyrene (ZnO/PS) nano-composite, a precipitated calcium carbonate, a carbon nano-tube structures, a silica nano-coating, a fluorinated silanes, or a fluoropolymer coating. The coating may include at least one of a hydrogel coating, an antimicrobial coating such as a silver ion coating and/or a chlorhexidine coating, an antibiotic coating, a medicinal coating, or any other coating suitable for minimizing HAI and improving patient comfort. According to one embodiment, the anti-microbial coating can be the BACTI-GUARD® Infection Prevention coating available from Bactiguard Holding AB. The plug body is suitably formed of a compliant material, including one of a polyvinyl chloride, a polyurethane, a “memory foam,” a silicone rubber, a foam rubber, a latex, or a compliant polymer. The urethral plug may further include a pull tab coupled to a proximal portion of the plug body and configured to facilitate grasping of the urethral plug.

Also disclosed herein is a method of mitigating urinary tract infections associated with a urinary catheter, including providing a urethral plug configured to engage the urinary catheter, having a plug body including a side surface, a proximal end, and a distal end configured to fit within a urethral opening, a lumen extending from the proximal end to the distal end and configured to receive the urinary catheter, and a slit extending longitudinally and extending from the lumen to a side surface of the plug body, inserting a portion of the urinary catheter through the slit, and to be received by the lumen of the plug body, and urging the plug body distally until a distal portion enters the urethral opening and the plug body creates a seal between the catheter and the urethral opening.

In some embodiments, a proximal portion is configured to remain outside of the urethral opening. The proximal end defines a first diameter, and the distal end defines a second diameter, the second diameter being less than the first diameter. The first diameter is between 3 mm and 30 mm, and the second diameter is between 2 mm and 10 mm. The first diameter is 10 mm, and the second diameter is 4 mm. The plug body further includes at least one of a textured surface and/or a coating disposed on a portion of a surface of the plug body, and configured to mitigate microbial adhesion. The textured surface includes one of a superomniphobic texture, a nano-cone texture, a superhydrophobic coating, a Manganese oxide polystyrene (MnO₂/PS) nano-composite, a Zinc oxide polystyrene (ZnO/PS) nano-composite, a precipitated calcium carbonate, a carbon nano-tube structures, a silica nano-coating, a fluorinated silanes, or a fluoropolymer coating. The coating includes at least one of a hydrogel coating, a silver ion coating, a chlorhexidine coating, an antibiotic coating, or a BACTI-GUARD® Silver Coating. The plug body is formed of a compliant material, including one of a polyvinyl chloride, a polyurethane, a “memory foam,” a silicone rubber, a foam rubber, a latex, or a compliant polymer. The plug body may further comprise a pull tab coupled to a proximal portion of the plug body and configured to facilitate grasping of the urethral plug.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of a urethral plug, in accordance with embodiments disclosed herein.

FIG. 2 shows a cross-sectional view of a urethral plug disposed within an opening, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

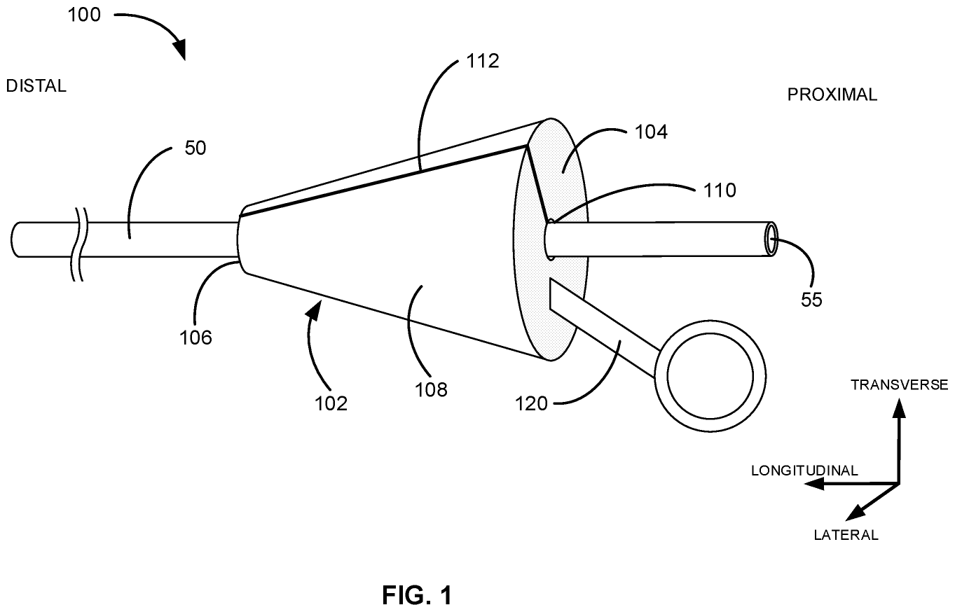

To assist in the description of embodiments described herein, as shown in FIG. 1, a longitudinal axis extends substantially parallel to an axial length of an insertion device 20. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

FIG. 1 shows a perspective view of an anti-microbial urethral plug (“urethral plug” or “plug”) 100 for the reduction of catheter associated urinary tract infections (“CAUTI”). The urethral plug 100 includes a plug body 102 that substantially defines a frustum shape, e.g. a frusto-conical shape, and includes a proximal end 104, a distal end 106, and a side surface 108 extending from the proximal end 104 to the distal end 106. The proximal end 104 and distal end 106 each define a substantially circular cross-sectional shape although it will be appreciated that other closed-curve, polygonal, cross-sectional shapes are also contemplated. Further, it will be appreciated that the side surface 108 of the plug body 102 can include one or more facets to define various polygonal frusta, (e.g. frusto-pyramid, frusto-pentagonal, and the like). The proximal end 104 defines a first diameter and the distal end 106 defines a second diameter, the second diameter being less than the first diameter. In an embodiment the proximal end 104 defines a diameter of between 3 mm and 30 mm, with a preferred embodiment having a diameter of 10 mm. In an embodiment the distal end 106 defines a diameter of between 2 mm and 10 mm, with a preferred embodiment having a diameter of 4 mm.

In an embodiment, the plug 100 further includes a lumen 110 extending axially from the proximal end 104 to the distal end 106 and sized to receive a urinary catheter 50 therethrough. In an embodiment the plug lumen 110 defines a diameter that is substantially the same, or slightly larger than, the diameter of the urinary catheter 50, to allow a urinary catheter 50 to fit snugly therein and without impacting the patency of the catheter lumen 55. In an embodiment the plug lumen 110 defines a diameter of between 2 mm and 10 mm, with a preferred embodiment having a diameter of 4 mm. However, it will be appreciated that plug lumen diameters greater or smaller than those disclosed are also contemplated.

In an embodiment, the diameter of the distal end 106 can be substantially the same as the diameter of the urinary catheter 50, such that the side surface 108 tapers from the diameter of the proximal end 104 to diameter of the urinary catheter 50. This creates a smooth transition between the surface of the catheter 50 and the side surface 108 of the plug body 102. In an embodiment, the plug 100 further includes a slit 112 extending longitudinally from the plug lumen 110, through the plug body 102 to an outer surface thereof and is configured to allow the catheter 50 to pass laterally or transversely therethrough.

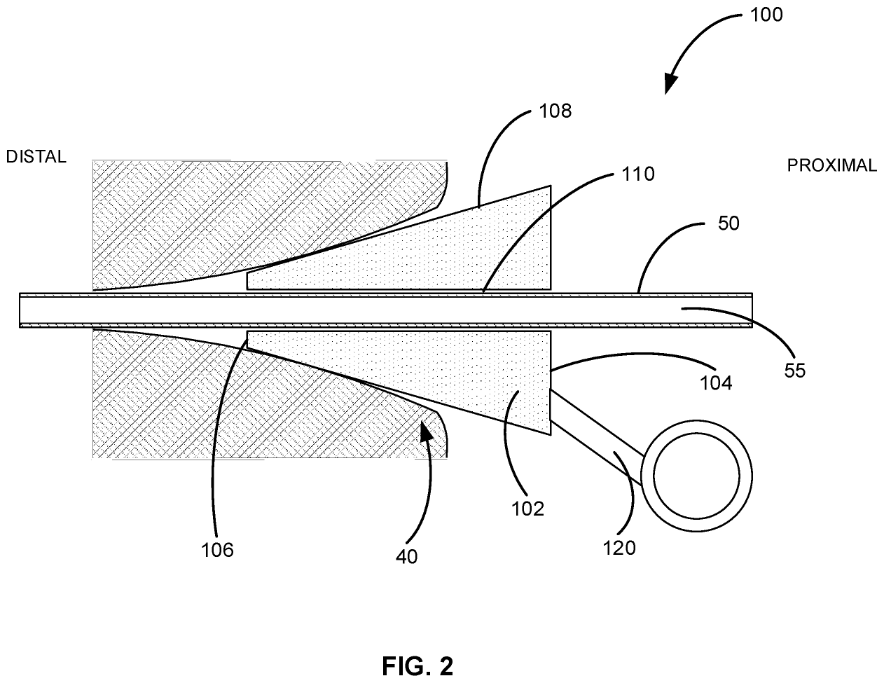

FIG. 2 shows a cross-sectional view of the urethral plug 100, including a urinary catheter 50, and disposed within an entrance of a urethra 40. The diameter of the distal end 106 is sized so that a distal portion of the plug 100 can fit within an entrance of the urethra 40. The diameter of the proximal end 104 is sized to prevent a proximal portion of the plug 100 from entering the urethra 40.

In an embodiment, the urethral plug 100 is formed of a compliant, biocompatible material. Exemplary materials include polyvinyl chloride (PVC), polyurethane (PU), “memory foam,” silicone rubber, foam rubber, latex, compliant polymers, combinations thereof, or the like. In an embodiment, the material that forms the urethral plug 100 includes anti-microbial compounds, medicinal compounds, or any other compounds suitable for minimizing HAIs and improving patient comfort. Exemplary anti-microbial compounds include silver ions, chlorhexidine, antibiotics, combinations thereof, or the like.

In an embodiment, a surface of the urethral plug 100 includes a textured surface that mitigates bacterial or microbial adhesion and/or growth. For example, a portion of the distal end 106, proximal end 104, the frusto-conical surface of the plug body 102, an inner surface of the plug lumen 110, or combinations thereof, includes one or more types of textured surface. Exemplary textured surfaces include “superomniphobic” textures, nano-cone textures, superhydrophobic coatings, Manganese oxide polystyrene (MnO₂/PS) nano-composite, Zinc oxide polystyrene (ZnO/PS) nano-composite, precipitated calcium carbonate, carbon nano-tube structures, silica nano-coating, fluorinated silanes and fluoropolymer coatings, combinations thereof, or the like. Advantageously, the textured surface can inhibit or mitigate bacterial or microbial growth and/or improve a user's grip on the urethral plug 100.

In an embodiment, a surface of the urethral plug 100 includes a coating that mitigates microbial adhesion and/or growth. Exemplary coatings include at least one of a hydrogel coating; an antimicrobial coating such as a silver ion coating and/or a chlorhexidine coating; an antibiotic coating, a medicinal coating, or any other coating suitable for minimizing HAIs and improving patient comfort. According to one embodiment, the anti-microbial coating can be the BACTI-GUARD® Infection Prevention coating available from Bactiguard Holding AB. In an embodiment, a surface of the urethral plug 100 includes a lubricious coating, for example, a hydrogel coating that activates when wetted, such as BARD® hydrogel. The lubricious coating can facilitate placement of the plug 100 within the urethra 40, sliding of the plug 100 relative to the catheter 50, or combinations thereof. According to one embodiment, the coating may include a pharmaceutically active coating for the delivery of at least one pharmaceutical to the urethral tissue.

In an embodiment, the plug 100 further includes a pull tab 120 extending proximally from a proximal portion of the urethral plug 100, e.g. proximal end 104, to facilitate manipulation or placement of the plug 100. In an embodiment, a user can grasp the pull tab 120 to facilitate withdrawal of the plug 100 from the urethra 40. Optionally, the pull tab 120 can include a finger loop, textured surface, or similar gripping feature to facilitate grasping the pull tab 120. The pull tab 120 can be formed from the same material as the plug 100. In an embodiment the pull tab 120 is formed of a different material from the plug 100, e.g. a foil.

The urethral plug 100 and catheter 50 described herein may be used in many different ways. A non-limiting method of use is described below. However, clinicians may vary the steps or procedures described herein, may reorder the steps, may perform additional steps beyond those described, and/or may omit certain steps as circumstances and a patient's unique needs may require. Further, the description below can be considered a description of an overall procedure with many steps or can be considered a series of individual methods or procedures each including only a subset of the steps described.

In an exemplary method of use, a urinary catheter 50 and associated catheterization kit can be provided for the insertion of a urinary catheter 50 into a patient. For example, the method may proceed in stages, i.e., (A) the initial peri-care stage, and (B) the catheterization stage. Further details and embodiments of catheterization kits and methods of use thereof can be found, in US 2017/0216558, which is incorporated by reference in its entirety into this application. The catheterization kit can include a perineal care (or peri-care) packet or kit. A peri-care kit is helpful to provide an initial cleaning of the area where the catheterization takes place prior to using an iodine solution to further cleanse and sanitize the area. The peri-care kit may include any items helpful for an initial cleaning of the patient's perineum or for perineum care in general. For example, the peri-care kit may include hand sanitizer (e.g., antiseptic gel hand rinse) for the health care provider to sanitize his/her hands, moist towelettes (e.g., a package of castile soap towelettes), instructions/procedural indicators (e.g., instructions/procedural indicators for health care provider and/or instructions for patient), and the like. The hand sanitizer may be designed to have improved efficacy and/or to enable single-handed usage.

Once the patient's perineal area is cleansed, the clinician can proceed with the catheterization stage. The catheterization stage can include the steps of, for example, i) unwrapping CSR wrap to create a sterile barrier, ii) donning sterile gloves, iii) placing an underpad under the patient's buttocks, the underpad including a waterproof side and a liquid absorbent side, iv) placing a fenestrated drape on the patient such that the genitalia are visible through the central opening in the fenestrated drape, and v) preparing the catheterization equipment for use. The catheterization kit can further include a package of antiseptic solution (e.g., povidone-iodine solution), swabs or swabsticks, or the like. The swabs/swabsticks can be soaked in the antiseptic solution in preparation for swabbing the genitalia through the central opening of the fenestrated drape. The catheterization kit can also include a lubricating jelly, or the like, that can be used to lubricate the catheter 50. The clinician can further attach a syringe of sterile liquid to the inflation port of the catheter 50, in preparation for inflating the catheter retention balloon.

The clinician can then proceed to cleanse the genitalia with the swabs/swabsticks. With the genitalia cleansed, the clinician can insert the lubricated catheter 50 into the urethra 40 to access the patient's bladder. Urine becomes visible in the tubing when the catheter 50 enters the bladder, but approximately two additional inches beyond this point should be inserted to allow the retention balloon to enter the bladder. The retention balloon can be inflated in the bladder by injecting the sterile liquid from the syringe of sterile liquid into the catheter through an inflation port. Once inflated, the clinician may gently pull the catheter 50 until the inflated balloon is snug against the bladder neck. These steps complete the catheterization stage.

With the urinary catheter 50 inserted into the patient, a portion of the shaft of the catheter 50 is inserted through the slit 112, into the lumen 110 of the urethral plug 100. The urethral plug 100 can be advanced distally along the catheter 50 until a distal end 106 enters the urethra 40. Optionally, the plug 100 and the catheter 50 can be advanced simultaneously. In an embodiment, the urethral plug 100 can be further advanced until a portion of the side surface 108 of the plug body 102 contacts the urethra 40. It is important to note that a proximal portion of the plug 100 remains outside of the urethra 40 to allow a user to grasp the plug 100. Accordingly, the plug 100 creates a seal between the outer surface of the catheter 50 and the inner surface of the entrance of the urethra 40. Advantageously, the plug 100 mitigates the introduction of pathogens, bacteria, microbes, or similar agents that can migrate along an outer surface of the urinary catheter, and cause CAUTI infections. As disclosed herein, the plug 100 can further include anti-microbial coatings and/or textures to further mitigate the introduction of pathogens that cause CAUTI infections.

Prior to removal of the urinary catheter 50, a proximal portion of the plug 100 can be grasped and withdrawn proximally. Optionally, the user can grasp the pull tab 120 to withdraw the plug proximally. The plug 100 can then be removed from the catheter 50 by passing the portion of the shaft of the catheter 50 through slit 112. The catheter 50 can then be removed from the patient. Optionally, the plug 100 can be removed simultaneously with the catheter 50.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A urethral plug configured to engage a urinary catheter and to mitigate urinary tract infection, the urethral plug comprising:
a plug body defining a frustum shape and comprising:
a lumen configured to receive the urinary catheter;
a slit extending longitudinally and extending from the lumen to a side surface of the plug body, the slit configured to allow the urinary catheter to pass therethrough;
at least one of a textured surface and a coating disposed on a portion of an outer surface and configured to mitigate microbial adhesion; and
a distal portion configured to fit within a urethral opening to create a seal between the side surface of the plug body and the urethral opening.

2. The urethral plug according to claim 1, further comprising a proximal portion configured to remain outside of the urethral opening.

3. The urethral plug according to claim 1, further comprising a proximal end defining a first diameter, and a distal end defining a second diameter, the second diameter being less than the first diameter.

4. The urethral plug according to claim 3, wherein the first diameter is between 3 mm and 30 mm, and the second diameter is between 2 mm and 10 mm.

5. The urethral plug according to claim 4, wherein the first diameter is 10 mm, and the second diameter is 4 mm.

6. The urethral plug according to claim 1, wherein the textured surface includes one of a superomniphobic texture, a nano-cone texture, a superhydrophobic coating, a Manganese oxide polystyrene (MnO₂/PS) nano-composite, a Zinc oxide polystyrene (ZnO/PS) nano-composite, a precipitated calcium carbonate, a carbon nano-tube structures, a silica nano-coating, a fluorinated silanes, or a fluoropolymer coating.

7. The urethral plug according to claim 1, wherein the coating includes at least one of an antimicrobial coating, an antibiotic coating, and a hydrogel coating.

8. The urethral plug according to claim 7, wherein the antimicrobial coating is selected from, a silver ion coating and a chlorhexidine coating.

9. The urethral plug according to claim 1, wherein the plug body is formed of a compliant material, including one of a polyvinyl chloride, a polyurethane, a “memory foam,” a silicone rubber, a foam rubber, a latex, or a compliant polymer.

10. The urethral plug according to claim 1, further comprising a pull tab coupled to a proximal portion of the plug body and configured to facilitate grasping of the urethral plug.

11. A method of mitigating urinary tract infections associated with a urinary catheter, comprising:
providing a urethral plug configured to engage the urinary catheter, comprising:
a plug body including a side surface, a proximal end, and a distal end configured to fit within a urethral opening;
a lumen extending from the proximal end to the distal end and configured to receive the urinary catheter; and
a slit extending longitudinally and extending from the lumen to a side surface of the plug body;
inserting a portion of the urinary catheter through the slit, and to be received by the lumen of the plug body; and
urging the plug body distally until a distal portion enters the urethral opening and the plug body creates a seal between the catheter and the urethral opening.

12. The method according to claim 11, wherein a proximal portion is configured to remain outside of the urethral opening.

13. The method according to claim 11, wherein the proximal end defines a first diameter, and the distal end defines a second diameter, the second diameter being less than the first diameter.

14. The method according to claim 13, wherein the first diameter is between 3 mm and 30 mm, and the second diameter is between 2 mm and 10 mm.

15. The method according to claim 14, wherein the first diameter is 10 mm, and the second diameter is 4 mm.

16. The method according to claim 11, wherein the plug body further includes one of a textured surface and a coating disposed on a portion of a surface of the plug body, and configured to mitigate microbial adhesion.

17. The method according to claim 16, wherein the textured surface includes one of a superomniphobic texture, a nano-cone texture, a superhydrophobic coating, a Manganese oxide polystyrene (MnO₂/PS) nano-composite, a Zinc oxide polystyrene (ZnO/PS) nano-composite, a precipitated calcium carbonate, a carbon nano-tube structures, a silica nano-coating, a fluorinated silanes, or a fluoropolymer coating.

18. The method according to claim 16, wherein the coating includes at least one of an antimicrobial coating, a hydrogel coating, and an antibiotic coating.

19. The method according to claim 18, wherein the antimicrobial coating is selected from a silver ion coating and a chlorhexidine coating.

20. The method according to claim 11, wherein the plug body is formed of a compliant material, including one of a polyvinyl chloride, a polyurethane, a “memory foam,” a silicone rubber, a foam rubber, a latex, or a compliant polymer.

21. The method according to claim 11, wherein the plug body further comprises a pull tab coupled to a proximal portion of the plug body and configured to facilitate grasping of the urethral plug.
